(19) United States
(12) Patent Application Publication
(10) Pub. No.: US 2009/0149708 A1
(43) Pub. Date: Jun. 11, 2009

(54) Treatment techniques using ingestible device

(76) Inventors: Roderick A. Hyde, Redmond, WA (US); Muriel Y. Ishikawa, Livermore, CA (US); Eric C. Leuthardt, St. Louis, MO (US); Michael A. Smith, Phoenix, AZ (US); Lowell L. Wood, JR., Bellevue, WA (US); Victoria Y.H. Wood, Livermore, CA (US)

Correspondence Address: BRAKE HUGHES BELLERMANN LLP, c/o CPA Global, P.O. BOX 52050, MINNEAPOLIS, MN, 55402, US

(21) Appl. No.: 12/006,255

(22) Filed: Dec. 31, 2007

Related U.S. Application Data
(63) Continuation of application No. 12/001,460, filed on Dec. 11, 2007.

(51) Int. Cl. A61B 1/00

(52) U.S. Cl. 600/118

(57) ABSTRACT

An apparatus, device, methods, computer program product, and systems are described that determine a control command at a patient internal device within a patient, and provide the control command to an ingestible device within the patient, the control command including instructions to the ingestible device to control an operation thereof.

### SUMMARY

An embodiment provides a method. In one implementation, the method includes but is not limited to determining a control command at a patient internal device within a patient, and providing the control command to an ingestible device within the patient, the control command including instructions to the ingestible device to control an operation thereof. In addition to the foregoing, other method aspects are described in the claims, drawings, and text forming a part of the present disclosure.

An embodiment provides a computer program product. In one implementation, the computer program product includes but is not limited to a signal-bearing medium bearing one or more instructions for determining a control command at a patient internal device within a patient. The signal bearing medium also may bear one or more instructions for providing the control command to an ingestible device within the patient, the control command including instructions to the ingestible device to control an operation thereof. In addition to the foregoing, other computer program product aspects are described in the claims, drawings, and text forming a part of the present disclosure.

In one or more various aspects, related systems include but are not limited to circuitry and/or programming for effecting the herein-referenced method aspects; the circuitry and/or programming can be virtually any combination of hardware, software, and/or firmware configured to effect the herein-referenced method aspects depending upon the design choices of the system designer.

An embodiment provides a system, the system including a computing device including computer-executable instructions that when executed on the computing device, cause the computing device to determine a control command at a patient internal device within a patient, and provide the control command to an ingestible device within the patient, the control command including instructions to the ingestible device to control an operation thereof. In addition to the foregoing, other system aspects are described in the claims, drawings, and text forming a part of the present disclosure.

An embodiment provides a system, the system comprising control logic configured to determine a control command at a patient internal device within a patient; and a transmitter configured to provide the control command to an ingestible device within the patient, the control command including instructions to the ingestible device to control an operation thereof. In addition to the foregoing, other system aspects are described in the claims, drawings, and text forming a part of the present disclosure.

An embodiment provides a method. In one implementation, the method includes but is not limited to receiving a control command from a patient internal device within a patient at an ingestible device within the patient, and controlling an operation of the ingestible device based on instructions within the control command. In addition to the foregoing, other method aspects are described in the claims, drawings, and text forming a part of the present disclosure.

An embodiment provides a computer program product. In one implementation, the computer program product includes but is not limited to a signal-bearing medium bearing one or more instructions for receiving a control command from a patient internal device within a patient at an ingestible device within the patient. The signal bearing medium also may bear one or more instructions for controlling an operation of the ingestible device based on instructions within the control command. In addition to the foregoing, other computer program product aspects are described in the claims, drawings, and text forming a part of the present disclosure.

An embodiment provides a system, the system including a computing device including computer-executable instructions that when executed on the computing device, cause the computing device to receive a control command from a patient internal device within a patient at an ingestible device within the patient, and control an operation of the ingestible device based on instructions within the control command. In addition to the foregoing, other system aspects are described in the claims, drawings, and text forming a part of the present disclosure.

An embodiment provides a system, the system comprising a receiver configured to receive a control command from a patient internal device within a patient at an ingestible device within the patient, and control logic configured to control an operation of the ingestible device based on instructions within the control command. In addition to the foregoing, other system aspects are described in the claims, drawings, and text forming a part of the present disclosure.

In addition to the foregoing, various other embodiments are set forth and described in the text (e.g., claims and/or detailed description) and/or drawings of the present description.

The foregoing is a summary and thus may contain simplifications, generalizations, inclusions, and/or omissions of detail; consequently, those skilled in the art will appreciate that the summary is illustrative only and is NOT intended to be in any way limiting. Other aspects, features, and advantages of the devices and/or processes and/or other subject matter described herein will become apparent in the teachings set forth herein.

### BRIEF DESCRIPTION OF THE DRAWINGS

FIG. 1 illustrates an example clinical system in which embodiments may be implemented to determine treatment using an ingestible device.

FIG. 2 illustrates an operational flow representing example operations related to determining treatments using an ingestible device.

FIG. 3 illustrates an alternative embodiment of the example operational flow of FIG. 2.

FIG. 5 illustrates an alternative embodiment of the example operational flow of FIG. 2.

FIG. 6 illustrates an alternative embodiment of the example operational flow of FIG. 2.

FIG. 7 illustrates an alternative embodiment of the example operational flow of FIG. 2.

FIG. 8 illustrates an alternative embodiment of the example operational flow of FIG. 2.

FIG. 9 illustrates an alternative embodiment of the example operational flow of FIG. 2.

FIG. 10 illustrates an alternative embodiment of the example operational flow of FIG. 2.

FIG. 11 illustrates a partial view of an example computer program product that includes a computer program for executing a computer process on a computing device.

FIG. 12 illustrates an example system in which embodiments may be implemented.

FIG. 13 illustrates another example operational flow representing example operations related to determining treatment using an ingestible device.

FIG. 14 illustrates a partial view of an example computer program product that includes a computer program for executing a computer process on a computing device.

The use of the same symbols in different drawings typically indicates similar or identical items.

### DETAILED DESCRIPTION

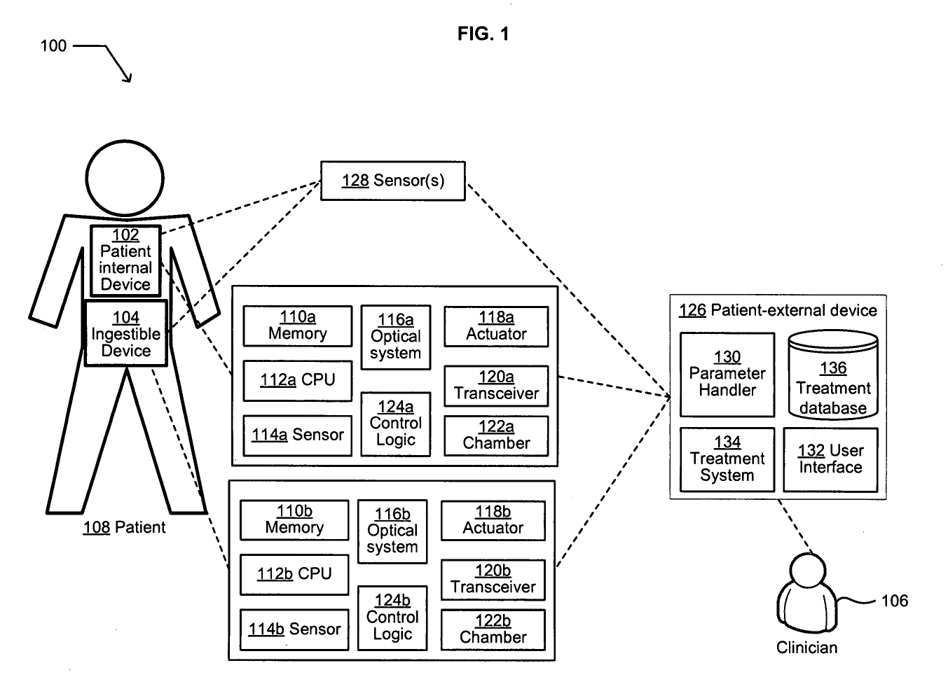

FIG. 1 illustrates an example clinical system 100 in which embodiments may be implemented to provide patient treatment using a patient internal device 102 and an ingestible device 104. The patient internal device 102, as described in more detail herein, may represent virtually any intracorporeal device that may be inserted, injected, ingested, or otherwise placed, e.g., by a clinician 106, within a patient 108. The patient internal device 102 may be used to provide a control command to the ingestible device 104, the control command including instructions to the ingestible device 104 to control an operation thereof. As described herein, such operation(s) may include, for example, a release of medication or other treatment agent by the ingestible device 104, a movement of the ingestible device 104, or virtually any other treatment-related operation of the ingestible device 104. Consequently, for example, treatment of the patient 108 may occur in a manner that is accurate, timely, reliable, flexible, and/or controllable. Further, the treatment(s) may occur in a manner that is on-going and non-invasive (except, e.g., for an initial placement of the patient internal device).

As referenced above, the patient internal device 102 may represent, for example, a pacemaker, stent, drug delivery device, or virtually any other intracorporeal device. As described in more detail herein, the patient internal device 102 may be placed virtually anywhere within a body of the patient 108 that is suitable for a desired function thereof, e.g., may be intravascular, may be placed internally by a suitable probe, scope, or needle, may be inhaled, may be implanted under the skin of the patient 108, may be provided as a suppository, or may be surgically or otherwise provided where desired within a bodily system (e.g., digestive, respiratory, or circulatory system(s)). Further, as described herein, the patient internal device 102 may itself be ingestible and may thus be swallowed by the patient 108.

The ingestible device 104 may represent, for example, virtually any device that may be swallowed by the patient 104. For example, as described herein, the ingestible device 104 may include a capsule or dispensing device that may be used to release medication or other treatment agent(s) within the patient 108. In example embodiments, the ingestible device 104 may generally take a pill form, but more generally may be any shape or size that allows ingestion by the patient 108. The ingestible device 104 (as with the patient internal device 102) may be associated with a size or construction material determined based on an intended use of the device(s) 102, 104. For example, the ingestible device 104 may be protected from digestion within the patient 108 (for later elimination by the patient 108), or, conversely, may be designed to be partially or wholly digested within the patient 108.

The clinician 106 may generally represent, for example, virtually any person involved in health care, including, for example, a doctor, a nurse, a physician's assistant, or a medical researcher. The clinician 106 also may represent someone who is involved in health care in the sense of developing, managing, or implementing the clinical system 100, e.g., a software developer with clinical knowledge (or access to clinical knowledge), a database manager, or an information technologies specialist. Even more generally, some or all of various functions or aspects described herein with respect to the clinician 106 may be performed automatically, e.g., by an appropriately-designed and implemented computing device, or by software agents or other automated techniques.

The patient 108 generally represents one or more persons with an illness, injury, or disease, or who is thought potentially to have such an illness, injury, or disease, or who may be wholly or partially healthy but who is nonetheless studied in order to determine information about such an illness, injury, or disease. The patient 108 also may represent or include other diagnostic and/or animal subjects that may be used in order, for example, to determine an efficacy of a particular medication or treatment, specific examples of which are provided herein. The patient 108 may represent a particular patient in a given clinical setting, such as in a doctor's office, or in a hospital, who is to be diagnosed and/or treated using the clinical system 100. The patient 108 also may represent the more abstract notion of a class of patients (e.g., patients having a certain age, gender, race, genetic makeup, or disposition to illness or disease), or, even more generally, may represent the general notion of a generic patient during basic research and/or development or application of various medical treatments or procedures. In the latter sense, the patient 108 also may represent a non-human animal (such as a primate) believed to be sufficiently similar to a human for the particular purposes that they may usefully substitute for such for the particular purposes.

In the example of FIG. 1, both the patient internal device 102 and the ingestible device 104 are illustrated as potentially including some or all of potential elements 110-122, where designators “a” and “b” are used for clarity when referring to the patient internal device 102 and the ingestible device 104, respectively. Consequently, in example embodiments, the patient internal device 102 and the ingestible device 104 may be essentially identical to one another in containing all (or some subset) of the elements 110-122. In other examples, various different combinations or subsets of the elements 110-122 (or other elements, not shown) may be included or associated with one or both of the patient internal device 102 and the ingestible device 104.

Thus, with example reference primarily to the elements 110a-122a of the patient internal device 102, memory 110a and central processing unit (CPU) 112a may represent virtually any such suitable elements that are of a size and performance level desired for carrying out desired ones of the various functions described herein, as well as many other functions that are not explicitly described. For example, the memory 110a may include flash memory and the CPU 112a may represent a microprocessor of an integrated circuit (microchip) that is integrated with, or in communication with, some or all of the remaining elements 114a-122a of the patient internal device 102.

A sensor 114a represents virtually any device or element that may be configured to measure, detect, determine, or otherwise sense information related to the patient 108. Many examples of the sensor 114a are provided herein, but, generally, the sensor 114a may sense information regarding to, for example, the patient's heart rate, blood pressure, or blood sugar, or any other biological, electrical, and/or chemical element or characteristic of the patient 108 that may be detected within the patient 108.

An optical system 116a may represent, for example, virtually any light-based or light-related system that may be useful to the clinician 106 in treating the patient 108. For example, the optical system 116a may represent an essentially passive element used to illuminate, record, or identify (e.g., using spectroscopy techniques) tissues or other portions within the patient 108, e.g., for diagnosis of the patient 108. In other example embodiments, the optical system 116a may represent a more active element that may be used in a treatment of the patient 108, such as a laser used for tissue ablation, binding, or separation.

An actuator 118a may represent one or more elements used by the patient internal device 102 to perform some function(s). Such functions may include, for example and as described herein, release of a medication or other treatment agent, collection of a tissue (or other) sample from the patient 108 if necessary, or control of a physical movement of the patient internal device 102 within the patient 108. For example, the actuator 118a may represent, in part, a motor or other element designed to impart motion to the patient internal device 102, e.g., to collect information using the optical system 116a, the sensor 114a, or some combination thereof.

Further in the patient internal device 102, a transceiver 120a represents virtually any transmitter and/or receiver (e.g., wireless or acoustic) that may be used to communicate with a device external to the patient internal device 102. Such devices may include, for example, the ingestible device 104, one or more external (e.g., extracorporeal) sensors 128, and/or a patient-external device 126 (e.g., used by the clinician 106 to communicate with one or both of the patient internal device 102 and the ingestible device 104).

A chamber 122a may represent virtually any opening, cavity, compartment, or otherwise hollowed or available portion of the patient internal device 102. The chamber 122a may thus be configured to store and/or dispense, e.g., medication or other treatment agent(s), and that may be configured (e.g., in response to an action of the actuator 118a, e.g., a mechanical or chemical pump) to release the desired medication or other treatment agent within the body of the patient 108.

Control logic 124a represents, for example, software, hardware, or combination(s) thereof, that may be used to control a behavior(s) of, e.g., the patient internal device 102 and/or the ingestible device 104. For example, the control logic 124a may obtain desired information from the sensor 114a (or 114b in the ingestible device), or from the sensor(s) 128, or from the patient-external device 126 of the clinician 106, or from other components of the patient internal device 102 or the ingestible device 104.

Then the control logic 124a may implement one or more algorithms to determine one or more behaviors of, for example, the patient internal device 102 or of the ingestible device 104. In particular, the control logic may transmit a control command (using the transceiver 120a) to the ingestible device, to control an operation thereof. For example, the control command may cause the ingestible device to release medication or other treatment agent(s) from the chamber 122b (e.g., antibiotics, chemotherapeutic agents, hormones, anti-coagulant agents, anti-proliferant agents, anti-inflammatory agents, steroids, or other appropriate medications), or may cause the ingestible device 104 to move within the patient 108 using the actuator 118b, e.g., to collect information using the optical system 116b.

As referenced herein, the control logic 124a also may operate based on, or in conjunction with, the patient-external device 126, in order to determine the control command for the ingestible device 104. The patient external device 126 may include, for example, a mobile computing device, such as a personal digital assistant (PDA), or a laptop computer. Of course, virtually any other computing device may be used, such as, for example, a workstation, a desktop computer, or a tablet PC.

The patient external device 126 may include a parameter handler 130 that may receive data from the sensor(s) 128, 114a, or 114b. A user interface 132, such as a graphical user interface, may be used by the clinician 106 to review the received parameters and to input additional information or instructions. A treatment system 134 may receive the parameters from the parameter handler 130 and/or the instructions or other information from the user interface 132, and may determine a preferred treatment option(s), perhaps using data from a treatment database 136. The treatment option(s) may then be sent to either or both of the patient internal device 102 and/or the ingestible device 104, either to control an operation(s) thereof, or to be used by control logic 124a, 14b as an input for use in determining a treatment option thereby.

Of course, in practice, not all of the components 130-136 need be implemented on a single computing device. For example, the parameter handler 130 and the user interface 132 may be implemented in part on a first device that is used locally by the clinician 106, while one or more of the treatment system 134 or the treatment database 136 may be stored and/or executed on a remote, networked device(s). In this way, the clinician 106, who may be operating in the field, e.g., in an office and/or hospital environment, may be relieved of a responsibility to update, manage, or manipulate the contents of the database 136, or other otherwise modify or update the treatment system 134, and may focus on determining proper treatment of the patient 108.

In an example operation, the patient internal device 102 and the ingestible device 104 may operate in closed-loop manner in which, for example, the ingestible device 104 collects information at the sensor 114b for transmission via the transceiver 120b to the control logic 124a of the patient internal device 102, which may then issue a control command to control an operation of the ingestible device 104. For example, the sensor 114b may collect data regarding blood sugar levels of the patient 108, and the control logic 124a may compute a necessary amount of insulin to be released by the chamber 122b of the ingestible device 104 (perhaps according to a pre-determined treatment model). In this example, it may occur that the sensor 128 detects some parameter associated with the patient 108 (e.g., increased/decreased heart rate or blood pressure, increased/decreased perspiration, or some other parameter(s)) and the patient external device 126 may then determine that a higher or lower dosage (e.g., of insulin) is needed than otherwise calculated by the control logic 124a, and may instruct the control logic 124a to modify (e.g., override) the control command to the ingestible device 104 accordingly. For example, in the example scenario referenced above, one or more set points of the treatment model may periodically be reset by the clinician 106, using the patient external device 126.

In some implementations, the chamber 122a may thus serve as a backup or redundant source of medication for the patient 108. For example, the clinician 104 may instruct the patient 108 to take a particular medication(s), such as medication to combat infections associated with human immunodeficiency virus (HIV). In these and other types of medications, it may be very important that the patient not miss a dose(s) of the medication. If the patient 108 does fail to ingest the ingestible device 104, or if the ingestible device 104 malfunctions (e.g., the chamber 122b fails to open), then the chamber 122a of the patient internal device 102 may provide a reserve dosage of the needed medication. Then, or in other cases where the patient internal device 102 and/or the ingestible device 104 determines that a dosage has been missed, a signal may be sent using one or both of the transceivers 120a, 120b, e.g., to the patient external device 126 and/or to a PDA or other device of the patient 108, that a dose has been missed and/or that the chamber 122a of the patient internal device 102 may (if feasible) need to be refilled.

In other examples, it may occur that a different dosage of a particular medication is required, depending on current symptoms or circumstances of the patient 108. For example, if the patient 108 suffers from diabetes, Parkinson's disease, or epilepsy, it may occur that only a portion of a medication dosage is necessary. In this case, the ingestible device 104 may contain a maximum amount of the medication within the chamber 122b, but, based on sensed data at the sensors 114a, 114b, or 128, or on other sources of information (e.g., patient-reporting), it may occur that the control command specifies some specific fraction of the available/maximum medication dosage actually be dispensed (e.g., released from the chamber 122b)

In some implementations, the patient internal device 102, e.g., the control logic 124a, may perform an analysis to see whether a desired medication (or the ingestible device 104 as a whole) is present within the patient 108, in order to provide the control command thereto. If the medication and/or ingestible device are not present (e.g., the medication within the chamber 122b has been used, or the ingestible device not yet swallowed, or the wrong ingestible device was swallowed), then the control logic 124a may signal (using the transceiver 120a) to the patient external device 126, in order, for example, to indicate to the clinician 106 and/or to the patient 108 that, e.g., additional ingestible device(s) should be ingested, and/or that the chamber 122a of the patient internal device should be refilled or replaced.

It will be appreciated in various embodiments that the illustrated components of FIG. 1 may be deployed in a wide variety of configurations. For example, the patient internal device 102 may contain some subset of the components 110a-124a, while the ingestible device 104 may contain some subset of the components 110b-124b. For example, the ingestible device 104 in some example embodiments may include only the chamber 122b, which may be entirely dependent on receiving the control command from the patient internal device 102. Conversely, it may occur that the ingestible device 104 includes all of the components 110b-124b, while the patient internal device 102 includes only sufficient structure to output a simple control command to the ingestible device 104, with the presumption that detailed treatment algorithms will be carried out at the control logic 124b of the ingestible device.

Meanwhile, the patient external device 126 may be a simple device that simply allows the clinician 106 to increase or decrease a dosage of medication from the chamber 122b. In these and other example embodiments, some or all of the illustrated components of the patient external device 126 may be included in, or associated with, the patient internal device 102. For example, the control logic 124a may include the parameter handler 130, the treatment system 134, and the treatment database 136, and may determine the control command to the ingestible device 104 based on input from some or all of the sensors 114a, 114b, 128, or from other inputs.

Thus, in various example embodiments, the patient internal device 102 and the ingestible device 104 may be virtually identical (e.g., may both be ingestible and contain the same or similar components), or may be quite different in terms of implementation and functionality. Meanwhile, the patient external device 126 also may have varying degrees of complexity, and may communicate directly with either or both of the patient internal device 102 and the ingestible device 104. Thus, it will be appreciated that the clinical system 100 of FIG. 1 provides many implementations for treating the patient 108 in a manner that improves a treatment of the patient 108 relative to conventional techniques, while minimizing an invasiveness of that treatment, minimizing side effects or other undesired outcomes, and minimizing an effort required of the patient 108 (e.g., to determine correct medications or dosages). Additional examples of the structure(s), and function(s) of the clinical system 100 of FIG. 1, and related systems, are provided herein.

Thus, it will be appreciated that FIG. 1 is not intended to provide a complete, detailed, or comprehensive set of examples of how the clinical system 100 may operate Rather, FIG. 1 merely provides a small number of selected examples, and additional and/or alternative examples are provided herein, as well. Further examples of implementation and use of the clinical system 100, and of related systems/techniques, also may be apparent.

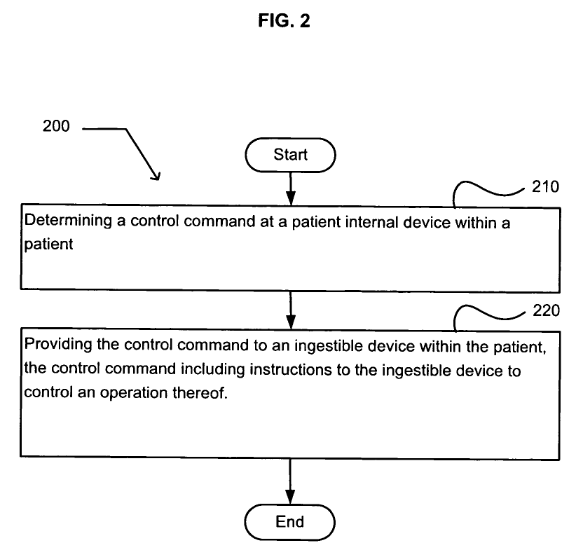

FIG. 2 illustrates an operational flow representing example operations related to determining treatments using an ingestible device. In FIG. 2 and in following figures that include various examples of operational flows, discussion and explanation may be provided with respect to the above-described examples of FIG. 1, and/or with respect to other examples and contexts. However, it should be understood that the operational flows may be executed in a number of other environments and contexts, and/or in modified versions of FIG. 1. Also, although the various operational flows are presented in the sequence(s) illustrated, it should be understood that the various operations may be performed in other orders than those which are illustrated, or may be performed concurrently.

After a start operation, the operational flow 200 moves to a determining operation 210, at which at least one control command is determined at a patient internal device within a patient. For example, the control logic 124a within the patient internal device 102 may determine the control command based on data received from one or more of the sensors 124a, 124b, or 128, and/or by implementing an algorithm to determine instructions to the ingestible device 104 to include within the control command.

In a providing operation 220, the control command may be provided to an ingestible device within the patient, the control command including instructions to the ingestible device to control an operation thereof. For example, the patient internal device 102 may provide the control command to the ingestible device 104, e.g., using the control logic 124a and/or the transceiver 120a. As described herein, the control command may include instructions to control operations of the ingestible device 104, including operations such as dispensing medication from the chamber 122b, moving to a desired location within the patient 108 using the actuator 118b, sensing data using the sensor 114b, or performing laser ablation using the optical system 116b.

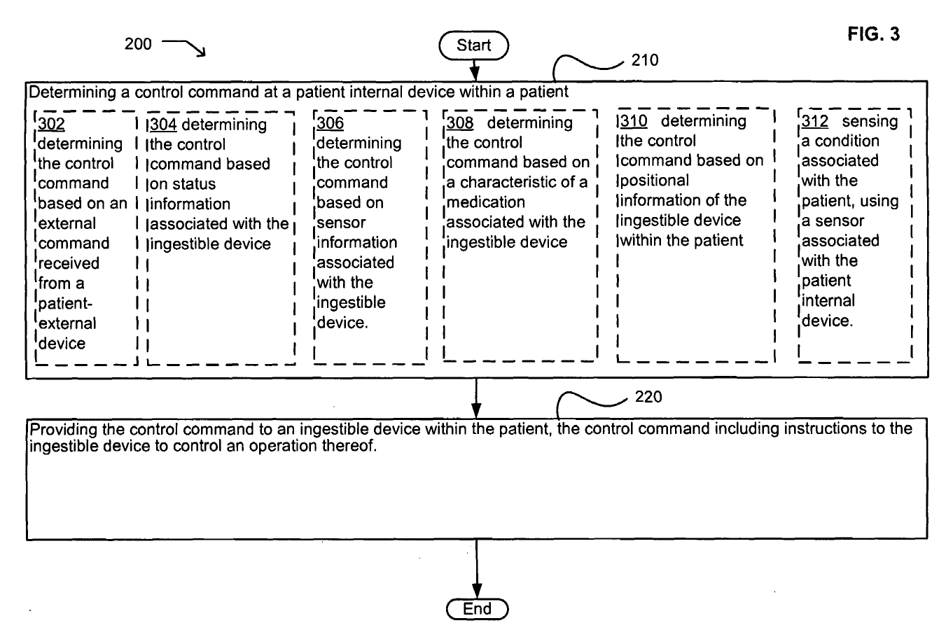

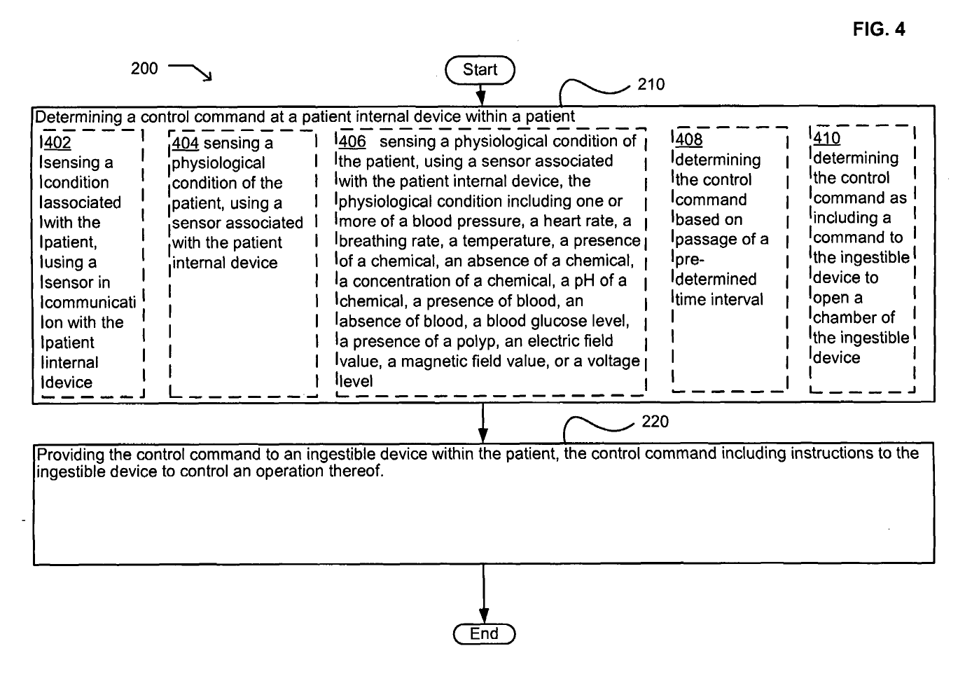

FIG. 3 illustrates alternative embodiments of the example operational flow 200 of FIG. 2. FIG. 3 illustrates example embodiments where the determining operation may include at least one additional operation. Additional operations may include an operation 302, an operation 304, an operation 306, an operation 308, and operation 310, and/or an operation 312.

At the operation 302, the control command may be determined based on an external command received from a patient-external device. For example, the transceiver 120a of the patient internal device 102 may receive such an external command from the patient-external device 126 of FIG. 1, and the control logic 124a of the patient internal device 102 may determine the control command based thereon, where the control command may include, for example, instructions to increase or decrease a dosage of medication released from the chamber(s) 122a, 122b, or may include many other types of instructions, e.g., as described herein.

At the operation 304, the control command may be determined based on status information associated with the ingestible device. For example, the transceiver 120a of the patient internal device 102 may receive a communication from the ingestible device 104, which may provide status information thereof, such as, for example, a current amount of medication or other treatment agent contained within the chamber 122b, a working condition of one of the components 110b-124b, or a location of the ingestible device 104 within the patient 108.

At the operation 306, the control command based may be determined on sensor information associated with the ingestible device. For example, the transceiver 120a of the patient internal device 102 may receive a communication from the ingestible device 104, based on data sensed by the sensor 114b. Similarly, the transceiver 120a may receive sensor information that is associated with the ingestible device 104, but that originates at least in part from a sensor that is external thereto. For example, the transceiver 120a may receive such sensor information from the sensor 114a or from the sensor 128, e.g., when one or more such sensors are in a vicinity of the ingestible device 104 within the patient.

At the operation 308, the control command may be determined based on a characteristic of a medication associated with the ingestible device. For example, the control logic 124a may determine the control command based on a characteristic of a medication within the chamber 122b of the ingestible device 104, where such a characteristic may include a quantity, a sufficiency of current quantity, a maximum or minimum quantity, or a type/profile of the medication (e.g., required dosages, or indication or contra-indications of usage based on other substances (e.g., other medications) present within the patient 108.

At the operation 310, the control command may be determined based on positional information of the ingestible device within the patient. For example, the control logic 124a may determine, based on sensor information received from the sensor 114b by way of the transceiver 120a, that the ingestible device is in a certain location within the patient 108, and may issue the control command according. For example, if the optical system 116b of the ingestible device includes a video transmission capability, then the control logic 124a may instruct the optical system 116b to begin transmitting video when near a desired viewing target (e.g., a potential location of a polyp, lesion, tumor, or other viewing target).

In another example embodiment, the control command may be determined based on patient-external information. For example, the patient-external device 126 may calculate a treatment to be implemented using the ingestible device 104, based on information available in the treatment database 136 (such as, e.g., the results of a clinical study).

In another example embodiment, the control command may be determined based on sensor information associated with the patient internal device. For example, the control logic 124a may determine the control command based on sensor information available from sensor(s) 114a, such as when the sensor 114a detects an increase in blood glucose levels of the patient 108, whereupon, as described herein, the control logic 124a may determine that the control command should include instructions to the ingestible device 104 to release insulin from the chamber 122b.

At the operation 312, a condition associated with the patient may be sensed, using a sensor associated with the patient internal device. For example, the sensor 114a may sense a condition of the patient 108, and the control logic 124a may determine the control command based at least partially thereon. For example, the patient internal device 102 may include a pacemaker, and the sensor 114a may sense a heart rate or other heart-related information for use by the control logic 124a in determining whether and to what extent medication should be released from the chamber 122b of the ingestible device 104.

FIG. 4 illustrates alternative embodiments of the example operational flow 200 of FIG. 2. FIG. 4 illustrates example embodiments where the determining operation 210 may include at least one additional operation. Additional operations may include an operation 402, an operation 404, an operation 406, an operation 408, and/or an operation 410.

At the operation 402, a condition associated with the patient may be sensed, using a sensor in communication with the patient internal device. For example, the sensor 128 may sense a condition of the patient 108, and the control logic 124a may determine the control command based at least partially thereon. For example, the sensor 128 may include virtually any external monitor of a condition of the patient 108, which may monitor vital statistics of the patient 108 such as heart rate, blood pressure, temperature, or respiration.

At the operation 404, a physiological condition of the patient may be sensed, using a sensor associated with the patient internal device. For example, the sensor 114a may sense a condition of the patient 108, and the control logic 124a may determine the control command based at least partially thereon. For example, similarly to the example above, the sensor 114a may sense a respiration rate of the patient 108, which may then be used by the control logic 124a to determine the control command.

At the operation 406, a physiological condition of the patient may be sensed, using a sensor associated with the patient internal device, the physiological condition including one or more of a blood pressure, a heart rate, a breathing rate, a temperature, a presence of a chemical, an absence of a chemical, a concentration of a chemical, a pH of a chemical, a presence of blood, an absence of blood, a blood glucose level, a presence of a polyp, an electric field value, a magnetic field value, or a voltage level. For example, as described, the sensor 114a may sense one or more of the parameters just mentioned, or other parameters not specifically set forth, and the control logic 124a may determine the control command base at least partially thereon.

At the operation 408, the control command may be determined based on passage of a pre-determined time interval. For example, the control logic 124a may determine that a pre-set time limit has passed since medication was last released from the chamber 122b, and may determine the control command (to cause the chamber 122b to release a dosage of the medication) based at least partially thereon.

At the operation 410, the control command may be determined as including a command to the ingestible device to open a chamber of the ingestible device. For example, the control logic 124a may, by way of the transceiver(s) 120a, 120b, instruct the chamber 122b to open to collect a sample from the patient 108, or to release a substance contained therein.

In another example embodiment, the control command may be determined as including a command to the ingestible device to promote a degradation of a surface of the ingestible device. For example, the control command may include a command that causes the control logic 124b of the ingestible device 104 to cause the actuator 118b to release a chemical from the chamber 122b that promotes a degradation of the surface of the ingestible device. For example, such degradation may be desirable to change a digestion characteristic of the ingestible device 104, e.g., either to speed or slow a digestion (or passage through the patient 108) of the ingestible device 104.

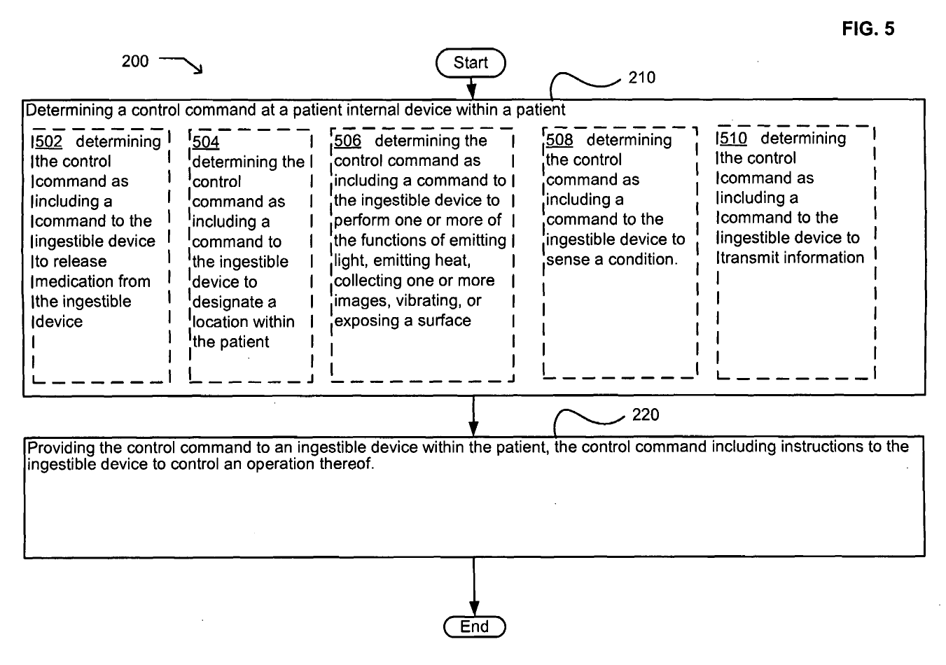

FIG. 5 illustrates alternative embodiments of the example operational flow 200 of FIG. 2. FIG. 5 illustrates example embodiments where the determining operation 210 may include at least one additional operation. Additional operations may include an operation 502, an operation 504, an operation 506, an operation 508, and/or an operation 510.

At the operation 502, the control command may be determined as including a command to the ingestible device to release medication from the ingestible device. For example, as just referenced, the control logic 124a may, by way of the transceiver(s) 120a, 120b, instruct the chamber 122b to open or otherwise release medication contained therein.

In another example embodiment, the control command may be determined as including a command to the ingestible device to release a specified amount of medication from the ingestible device. For example, the control logic 124a may, by way of the transceiver(s) 120a, 120b, instruct the chamber 122b to open or otherwise release a specified dosage of medication contained therein.

In another example embodiment, the control command may be determined as including a command to the ingestible device to stop a motion thereof. For example, the control command may cause the control logic 124b to instruct the actuator 118b to move counter to any motion induced by the digestive tract of the patient 108, or to attach or otherwise maintain position with respect to a body part of the patient 108 (e.g., to dispense medication at the position).

At the operation 504, the control command may be determined as including a command to the ingestible device to designate a location within the patient. For example, the control logic 124a may, by way of the transceiver(s) 120a, 120b, instruct the actuator 118b to mark a location within the patient for later administration(s) of medication, or for later identification/observation of the identified location. For example, the actuator 118b may leave a chemical or mechanical maker at a location of a possible polyp, tumor, or other portion of the patient 108.

At the operation 506, the control command may be determined as including a command to the ingestible device to perform one or more of the functions of emitting light, emitting heat, collecting one or more images, vibrating, or exposing a surface. For example, the control logic 124a may, by way of the transceiver(s) 120a, 120b, instruct the optical system 116 and/or the actuator 118b, or similar or related components, to perform one or more of the function just referenced.

At the operation 508, the control command may be determined as including a command to the ingestible device to sense a condition. For example, For example, the control logic 124a may, by way of the transceiver(s) 120a, 120b, instruct the sensor 114b to take a measurement or otherwise obtain data from a current location of the ingestible device 104.

At the operation 510, the control command may be determined as including a command to the ingestible device to transmit information. For example, the control logic 124a may, by way of the transceiver(s) 120a, 120b, instruct the control logic 124b of the ingestible device to perform some algorithm or calculation (e.g., aggregating sensed data over a period of time) for transmission or subsequent results to the patient internal device 102.

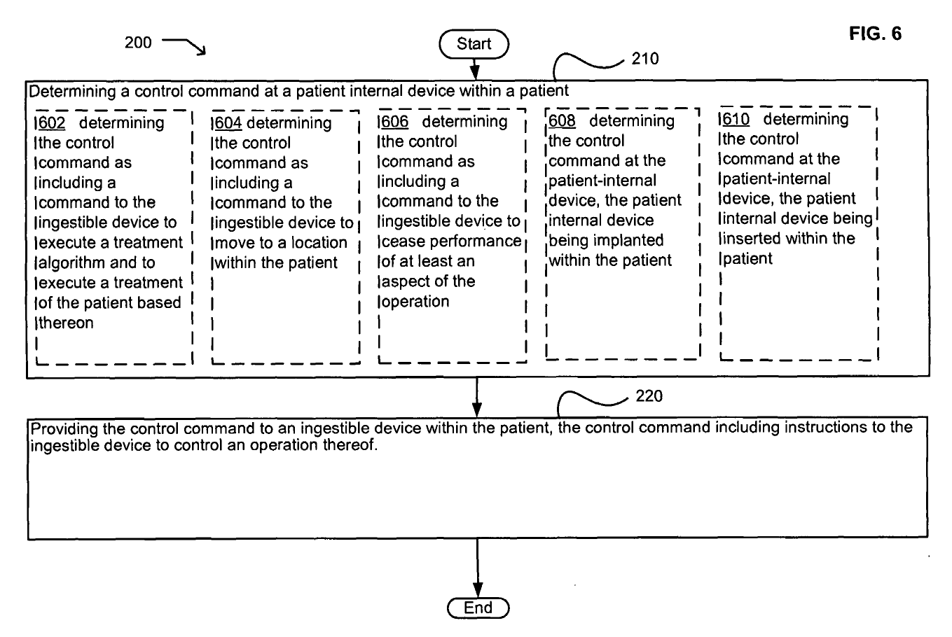

FIG. 6 illustrates alternative embodiments of the example operational flow 200 of FIG. 2. FIG. 6 illustrates example embodiments where the receiving operation 210 may include at least one additional operation. Additional operations may include an operation 602, an operation 604, an operation 606, an operation 608, and/or an operation 610.

At the operation 602, the control command may be determined as including a command to the ingestible device to execute a treatment algorithm and to execute a treatment of the patient based thereon. For example, the control logic 124b may receive the control command as a simple instruction to begin local treatment calculations/determinations, whereupon the control logic 124b may proceed to gather information (e.g., from the sensor 114b or the optical system 116b) and to execute an algorithm based thereon to make a treatment decision for the patient 108 (e.g., whether to stop/start or increase/decrease medication released from the chamber 122b). In this regard, the control logic 124b may be considered to implement some or all of the parameter handler 130, the treatment database 136, and the treatment system 134, in order to locally determine treatment options for the patient 108.

At the operation 604, the control command may be determined as including a command to the ingestible device to move to a location within the patient. For example, the control logic 124a may determine from the sensor 114a, the optical system 116a, or the sensor 128, that medication should be dispensed, or the patient 108 should be observed/treated, at a certain location within, e.g., the digestive tract of the patient 108. Then, the control command may include instructions to the actuator 118b to move the ingestible device 104 to the determined location.

At the operation 606, the control command may be determined as including a command to the ingestible device to cease performance of at least an aspect of the operation. For example, in an example where the ingestible device has previously been instructed to release medication from the chamber 122b, the control command may include instructions to the control logic 124b to stop releasing the medication (e.g., due to possible overdose or allergic reaction). In other examples, the control logic 124a may determine a (possible) presence of alcohol, illicit drugs, or contraindicated drugs within the patient 108, and may proactively stop or prevent release of medication from the chamber 122b in order to prevent negative effects to the patient 108. For example, although the clinician 106 is illustrated as operating the patient external device in FIG. 1, it may occur that the patient 108 is provided with the patient external device 126, so as to prevent some or all of the negative effects referenced above or elsewhere herein.

At the operation 608, the control command may be determined at the patient internal device, the patient internal device being implanted within the patient. For example, the patient internal device 102 may be implanted within the patient 108, e.g., in or on a brain, heart or other organ of the patient 108.

At the operation 610, the control command may be determined at the patient internal device, the patient internal device being inserted within the patient. For example, the patient internal device 102 may be inserted endoscopically, or by injection, e.g., by subcutaneous/intramuscular/subdermal injection.

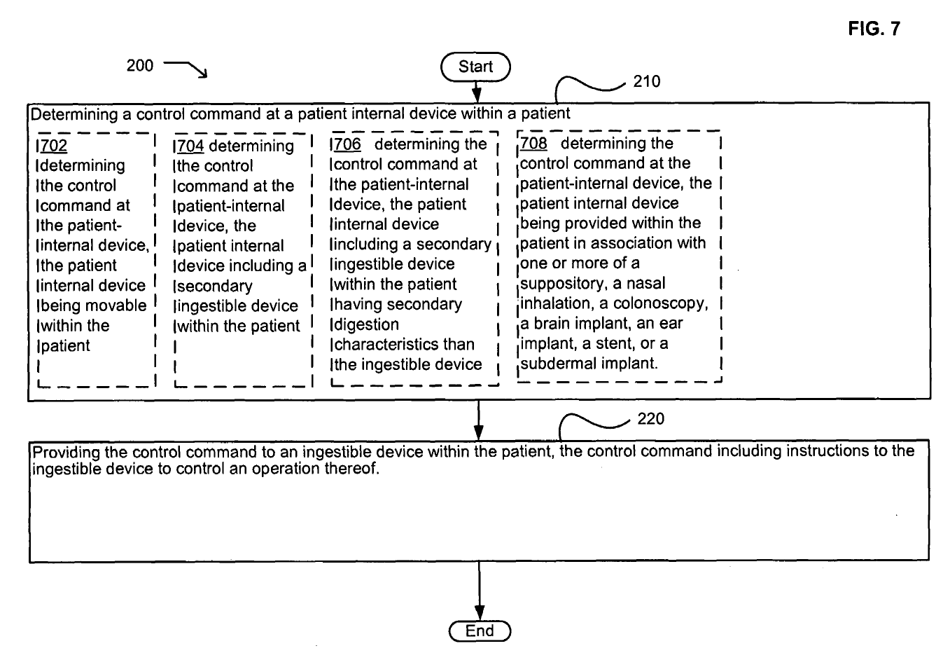

FIG. 7 illustrates alternative embodiments of the example operational flow 200 of FIG. 2. FIG. 7 illustrates example embodiments where the determining operation 210 may include at least one additional operation. Additional operations may include an operation 702, an operation 704, an operation 706, and/or an operation 708.

At the operation 702, the control command may be determined at the patient internal device, the patient internal device being movable within the patient. For example, the patient internal device 102 may be relocated by the clinician 106, e.g., using an appropriate scope, or the actuator 118a may provide varying levels of independent movement within the patient 108.

At the operation 704, the control command may be determined at the patient internal device, the patient internal device including a secondary ingestible device within the patient. For example, as illustrated in FIG. 1, the patient internal device 102 and the ingestible device 104 may structurally be the same or similar, and, for example, may be substantially identical devices which are both ingested (at the same or different times) with different instructions, functions, or purposes (e.g., containing different medications, or containing a different release schedule of the medication(s)).

At the operation 706, the control command may be determined at the patient internal device, the patient internal device including a secondary ingestible device within the patient having secondary digestion characteristics than the ingestible device. For example, as just referenced, the patient internal device 102 and the ingestible device 104 may be similar or identical to one another, except that, upon ingestion, one of the devices 102, 104 may be digested differently. For example, different coatings may be used that are associated with digestion in different portions of the digestive tract of the patient 108. In other examples, different coatings of, or attachments to, one of the devices 102, 104 may cause a relative delay of one of the devices 102, 104 through the digestive tract.

At the operation 708, the control command may be determined at the patient internal device, the patient internal device being provided within the patient in association with one or more of a suppository, a nasal inhalation, a colonoscopy, a brain implant, an ear implant, a stent, or a subdermal implant. For example, one or more of the patient internal device(s) 102 may be placed internally within the patient 108 using one or more of the just-referenced techniques.

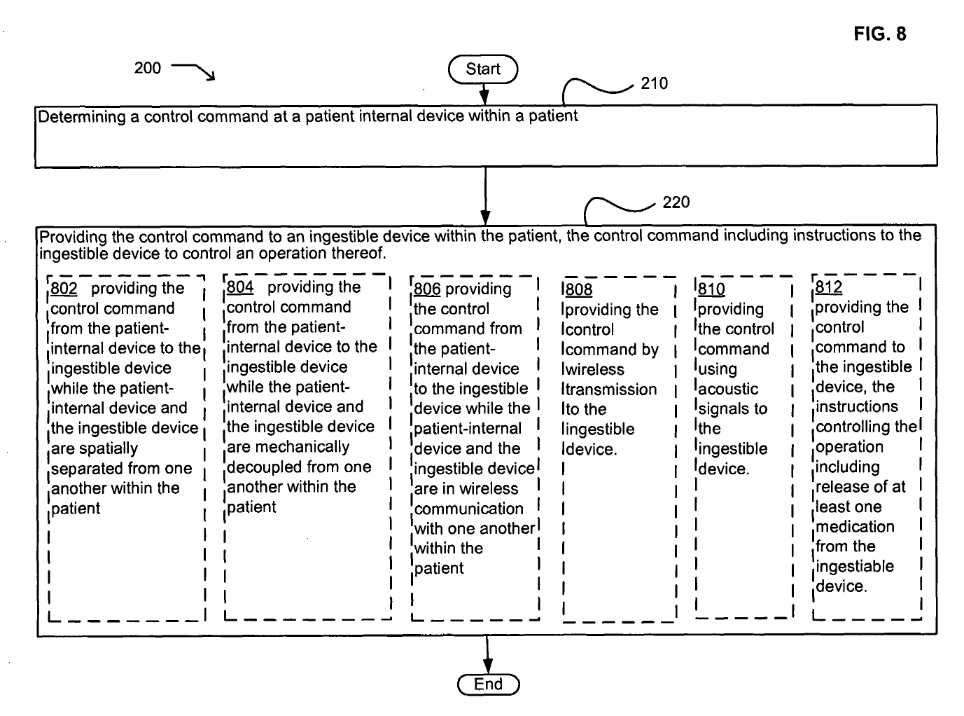

FIG. 8 illustrates alternative embodiments of the example operational flow 200 of FIG. 2. FIG. 8 illustrates example embodiments where the providing operation 220 may include at least one additional operation. Additional operations may include an operation 802, an operation 804, an operation 806, and operation 808, an operation 810, and/or an operation 812.

At the operation 802, the control command may be provided from the patient internal device to the ingestible device while the patient internal device and the ingestible device are spatially separated from one another within the patient. For example, the patient internal device 102 may include a pacemaker or brain implant, while the ingestible device 104 may be contained within a digestive tract of the patient 108.

At the operation 804, the control command may be provided from the patient internal device to the ingestible device while the patient internal device and the ingestible device are mechanically decoupled from one another within the patient. For example, the patient internal device 102 and the ingestible device 104, as referenced above, may both be ingestible, but may travel through the digestive tract of the patient 108 independently of one another, with no mechanism for coupling or attaching to one another within the digestive tract.

At the operation 806, the control command may be provided from the patient internal device to the ingestible device while the patient internal device and the ingestible device are in wireless communication with one another within the patient. For example, the transceivers 120a and 120b may enable wireless communication between the patient internal device 102 and the ingestible device 104.

At the operation 808, the control command may be provided by wireless transmission to the ingestible device. For example, the control logic 124a may cause the transceiver 120a to transmit the control command to the ingestible device 104.

At the operation 810 the control command may be provided using acoustic signals to the ingestible device. For example, the transceivers 120a, 120b may represent, or be associated with, transducers configured to transmit/receive acoustic signals using available acoustic media (e.g., fluids) within the digestive tract of the patient 108.

At the operation 812, the control command may be provided to the ingestible device, the instructions controlling the operation including release of at least one medication from the ingestible device. For example, as described herein, the control logic 124a may provide the control command as including instructions to the ingestible device 104 to release medication from the chamber 122b.

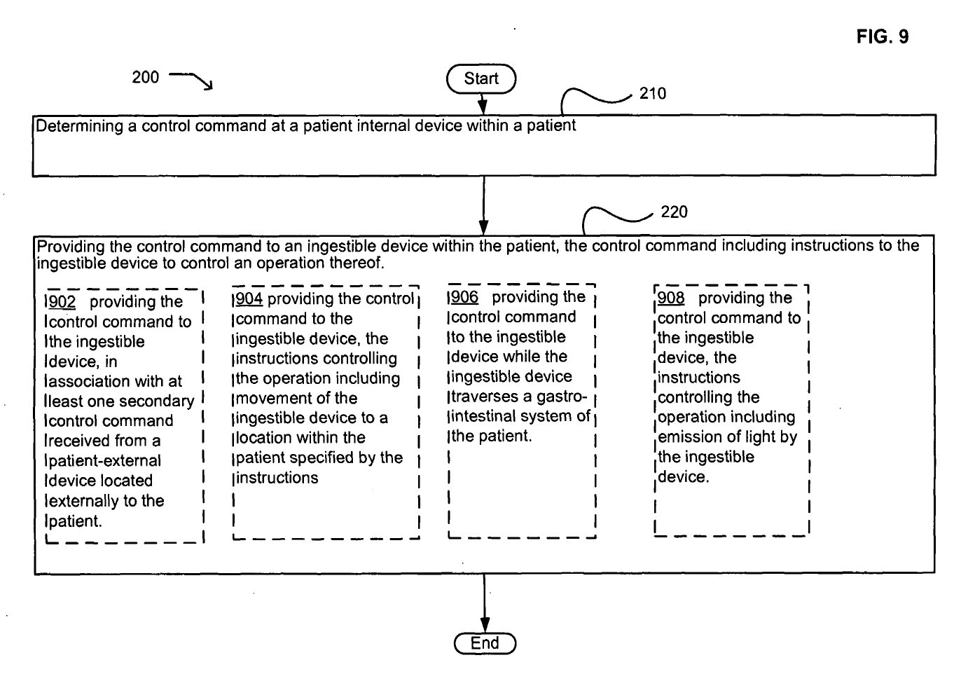

FIG. 9 illustrates alternative embodiments of the example operational flow 200 of FIG. 2. FIG. 9 illustrates example embodiments where the providing operation 220 may include at least one additional operation. Additional operations may include an operation 902, an operation 904, an operation 906, and/or an operation 908.

At the operation 902, the control command may be provided to the ingestible device, in association with at least one secondary control command received from a patient-external device located externally to the patient. For example, the control command may be provided to the ingestible device 104 in conjunction with a secondary control command from the patient-external device 126. For example, the control command may include a command to release medication from the chamber 122b, but the secondary control command may override this command at a discretion of the clinician 106 (e.g., to raise, lower, or eliminate the dose).

At the operation 904, the control command may be provided to the ingestible device, the instructions controlling the operation including movement of the ingestible device to a location within the patient specified by the instructions. For example, the control logic 124a may determine that an observation or treatment may be needed at a location within the patient 108, and may instruct the actuator 118b to move the ingestible device 104 to the location.

At the operation 906, the control command may be provided to the ingestible device while the ingestible device traverses a gastro-intestinal system of the patient. For example, the patient internal device 102 may provide the control command to the ingestible device 104 while the ingestible device is in transit within the gastro-intestinal system of the patient 108, e.g., different control commands may be provided to the ingestible device 104 while it is in the stomach as opposed to while it is within the small intestine of the patient 108.

At the operation 908, the control command may be provided to the ingestible device, the instructions controlling the operation including emission of light by the ingestible device. For example, the control logic 124a may determine that an observation of a location within the patient 108 is needed, and may activate the optical system 116b to record and/or transmit such observation(s) of the specified location, using the optical system 116b. In other, already-described implementations, the instructions also may include use of the optical system 116b as a laser or other optical tool for actively performing treatment on the patient 108.

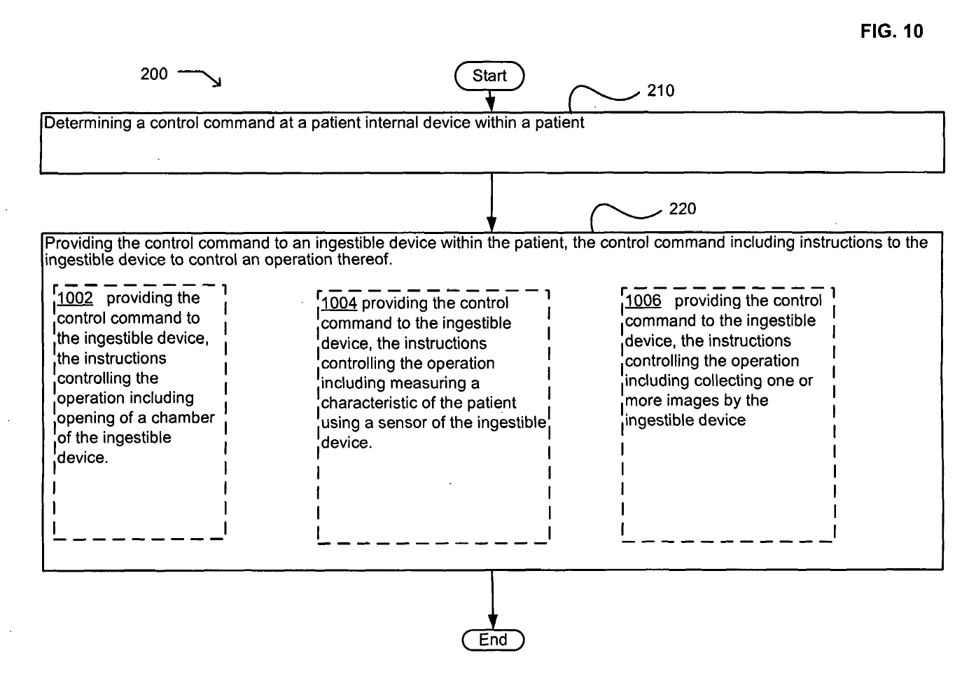

FIG. 10 illustrates alternative embodiments of the example operational flow 200 of FIG. 2. FIG. 10 illustrates example embodiments where the providing operation 220 may include at least one additional operation. Additional operations may include an operation 1002, an operation 1004, and/or an operation 1006.

At the operation 1002, the control command may be provided to the ingestible device, the instructions controlling the operation including opening of a chamber of the ingestible device. For example, the control logic 124a may determine the control command including instructions to the control logic 124b of the ingestible device to open the chamber 122b of the ingestible device.

At the operation 1004, the control command may be provided to the ingestible device, the instructions controlling the operation including measuring a characteristic of the patient using a sensor of the ingestible device. For example, the control logic 124a may instruct the sensor 114b (directly or by way of the control logic 124b) to measure a presence or concentration of a chemical within a gastro-intestinal system of the patient 108.

At the operation 1006, the control command may be provided to the ingestible device, the instructions controlling the operation including collecting one or more images by the ingestible device. For example, the control logic 124a may determine that an observation of a location within the patient 108 is needed, and may activate the optical system 116b to record and/or transmit picture(s) or video of the specified location, using the optical system 116b.

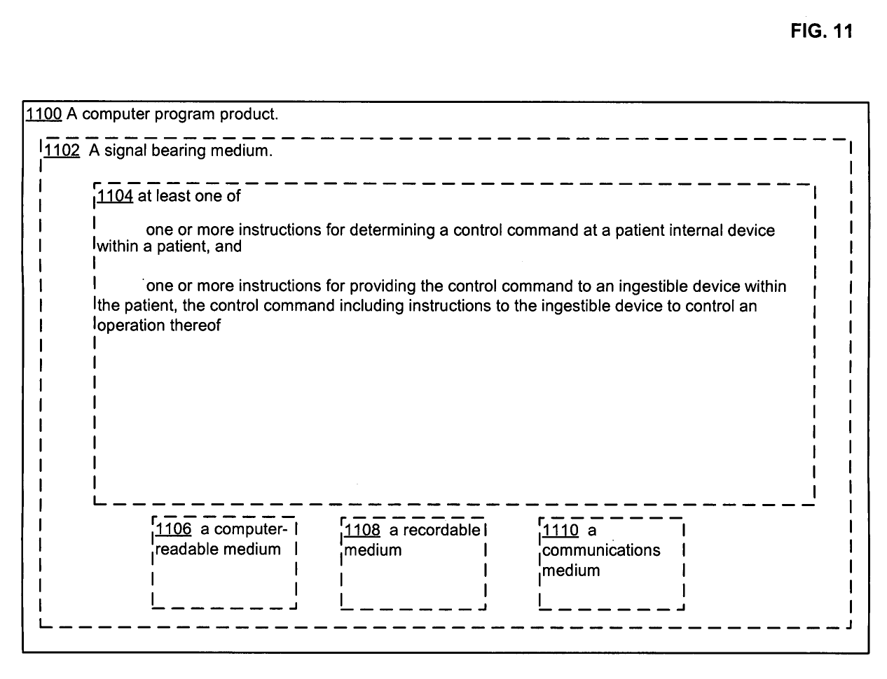

FIG. 11 illustrates a partial view of an example computer program product 1100 that includes a computer program 1104 for executing a computer process on a computing device. An embodiment of the example computer program product 1100 is provided using a signal bearing medium 1102, and may include one or more instructions for determining a control command at a patient internal device within a patient. The signal bearing medium 1102 also may bear one or more instructions for providing the control command to an ingestible device within the patient, the control command including instructions to the ingestible device to control an operation thereof.

The one or more instructions may be, for example, computer executable and/or logic-implemented instructions. In one implementation, the signal-bearing medium 1102 may include a computer-readable medium 1106. In one implementation, the signal bearing medium 1102 may include a recordable medium 1108. In one implementation, the signal bearing medium 1102 may include a communications medium 1110.

For example, the computer program product 1100 may be used as, or in conjunction with, the control logic 124a, which may implement the computer executable and/or logic-implemented instructions to determine the control command (including the instructions to the ingestible device) and provide the control command to the ingestible device.

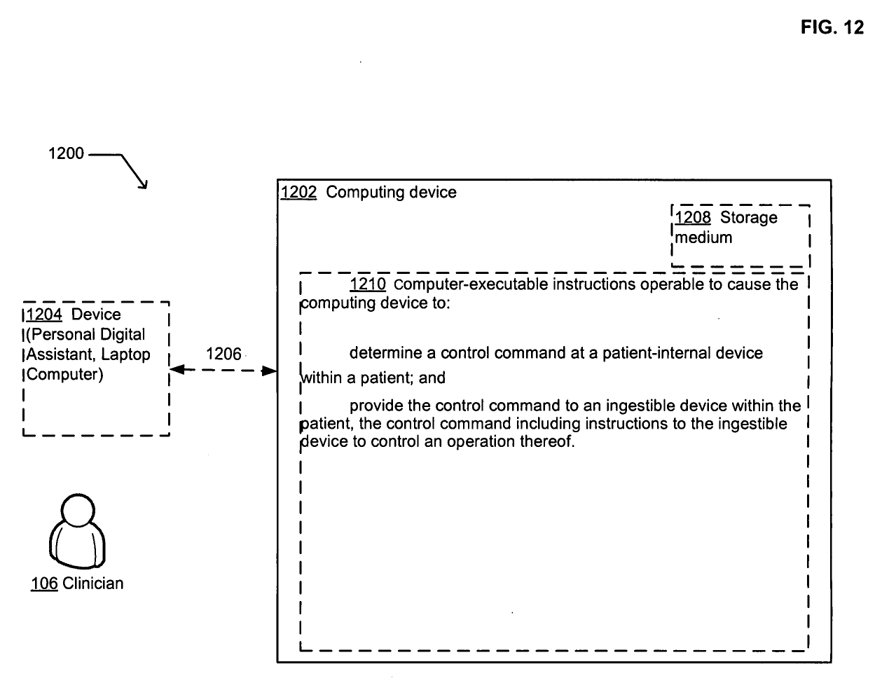

FIG. 12 illustrates an example system 1200 in which embodiments may be implemented. The system 1200 includes a computing system environment. The system 1200 also illustrates the clinician 106 using a device 1204, which is optionally shown as being in communication with a computing device 1202 by way of an optional coupling 1206. For example, the computing device 1204 may represent the patient external device 126, while the computing device 1202 may represent the patient internal device 102. The optional coupling 1206 may represent a local, wide-area, or peer-to-peer network that is formed between the patient external device and a plurality of patient internal devices (including ingestible device(s)). A storage medium 1208 may be any computer storage media, e.g., represented as the memory 110a of FIG. 1.

The computing device 1202 includes computer-executable instructions 1210 that when executed on the computing device 1202, cause the computing device 1202 to determine a control command at a patient internal device within a patient, and provide the control command to an ingestible device within the patient, the control command including instructions to the ingestible device to control an operation thereof.

In FIG. 12, then, the system 1200 includes at least one computing device (e.g., 1202 and/or 1204). The computer-executable instructions 1210 may be executed, for example, on the computing device 1202, and additional or related instructions may be computed on the device 1204. The clinician device 1204 may include, for example and as referenced above with respect to the patient external device 126, one or more of a personal digital assistant (PDA), a laptop computer, a tablet personal computer, a networked computer, a computing system comprised of a cluster of processors, a workstation computer, and/or a desktop computer.

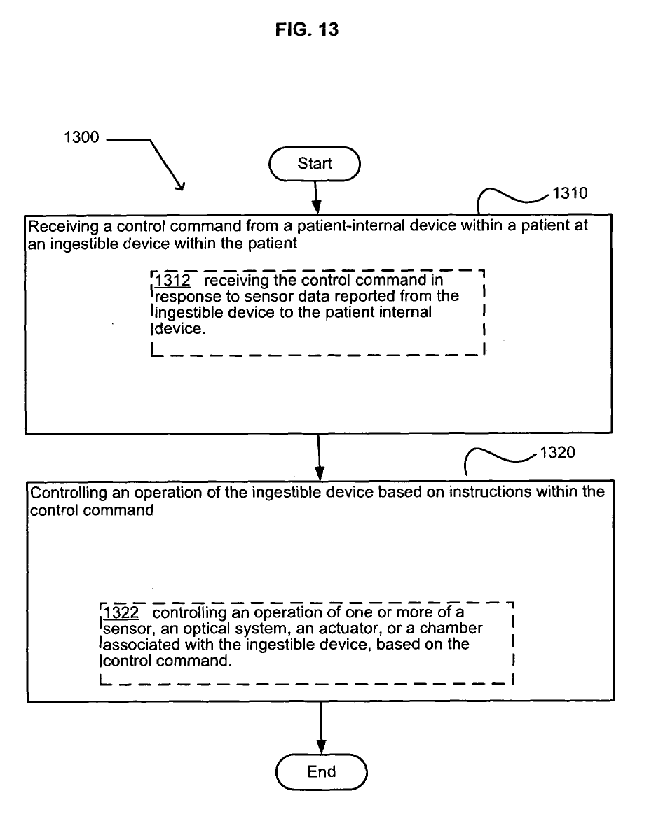

FIG. 13 illustrates another example operational flow representing example operations related to diagnosis through graphical representations of patient characteristics. In FIG. 13 and related various examples of operational flows, discussion and explanation may be provided with respect to the above-described examples of FIGS. 1, and/or with respect to other examples and contexts. However, it should be understood that the operational flow(s) may be executed in a number of other environments and contexts, and/or in modified versions of FIG. 1. Also, although the various operational flows are presented in the sequence(s) illustrated, it should be understood that the various operations may be performed in other orders than those which are illustrated, or may be performed concurrently.

After a start operation, the operational flow 1300 moves to a receiving operation 13 10, in which a control command may be received from a patient internal device within a patient at an ingestible device within the patient. For example, as described herein, the control command may be received at a transceiver of the ingestible device 104 from the patient internal device 102.

In a controlling operation 1320, an operation of the ingestible device may be controlled, based on instructions within the control command. For example, the control logic 124b may control the chamber 122b to open and dispense medication, or may instruct the sensor 114b to obtain sensed data, or may instruct the actuator 116b to move the ingestible device or take other action.

FIG. 13 also illustrates alternative embodiments of the example operational flow 1300 of FIG. 13. Thus, FIG. 13 illustrates example embodiments where the receiving operation 1310 may include at least one additional operation (e.g., the operation 1312), and the controlling operation 1320 may include at least one additional operation (e.g., the operation 1322).

For example, at the operation 1312, the control command may be received in response to sensor data reported from the ingestible device to the patient internal device. For example, data from the sensor 114b may be reported to the patient internal device 102, which may then determine (e.g., using the control logic 124a) the control command, thereby forming a closed operational/feedback loop between the patient internal device 102 and the ingestible device 104. At the operation 1322, an operation of one or more of a sensor, an optical system, an actuator, or a chamber associated with the ingestible device may be controlled, based on the control command. For example, the sensor 114b may be used to report blood glucose levels of the patient 108, which may be reported to the control logic 124a of the patient internal device, which may itself then provide the control command as including instructions to release insulin from the chamber 122b.

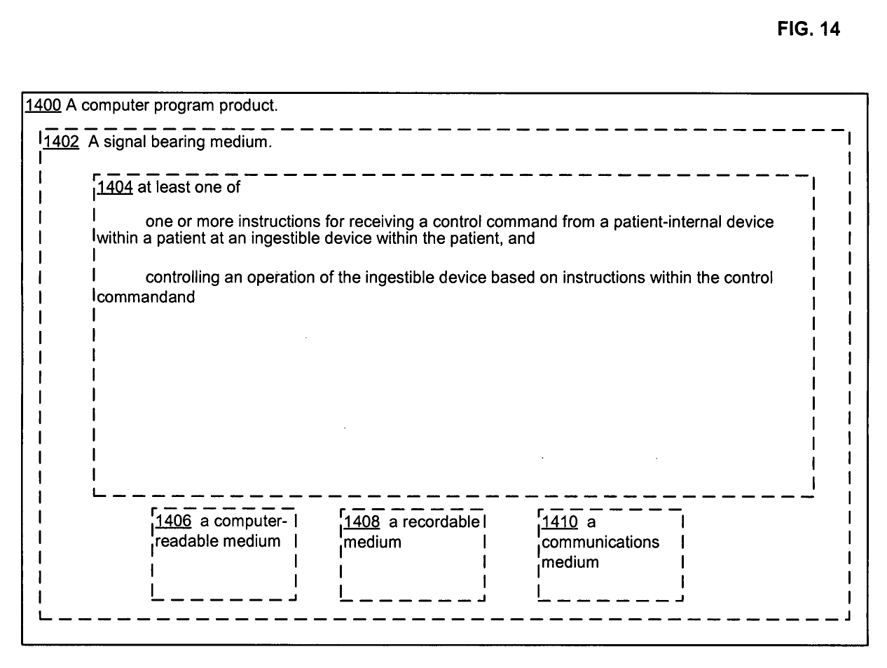

FIG. 14 illustrates a partial view of an example computer program product that includes a computer program for executing a computer process on a computing device. FIG. 14 illustrates a partial view of an example computer program product 1400 that includes a computer program 1404 for executing a computer process on a computing device. An embodiment of the example computer program product 1400 is provided using a signal bearing medium 1402, and may include one or more instructions for receiving a control command from a patient internal device within a patient at an ingestible device within the patient. The signal bearing medium 1402 also may bear one or more instructions. The signal bearing medium 1402 also may bear one or more instructions for controlling an operation of the ingestible device, based on instructions within the control command.

The one or more instructions for controlling an operation of the ingestible device 104 may include, for example, computer executable and/or logic-implemented instructions. In one implementation, the signal-bearing medium 1402 may include a computer-readable medium 1406. In one implementation, the signal bearing medium 1402 may include a recordable medium 1408. In one implementation, the signal bearing medium 1402 may include a communications medium 1410. For example, the computer program product 1400 may be used as, or in conjunction with, the control logic 124b, which may implement the computer executable and/or logic-implemented instructions to respond to the received control command and determine control an operation of the ingestible device 104 based thereon.

Those having skill in the art will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link, etc.).

In a general sense, those skilled in the art will recognize that the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermediate components. Likewise, any two components so associated can also be viewed as being “operably connected,” or “operably coupled,” to each other to achieve the desired functionality. Any two components capable of being so associated can also be viewed as being “operably couplable” to each other to achieve the desired functionality. Specific examples of operably couplable include but are not limited to physically mateable and/or physically interacting components and/or wirelessly interactable and/or wirelessly interacting components and/or logically interacting and/or logically interactable components.

While particular aspects of the present subject matter described herein have been shown and described, it will be apparent to those skilled in the art that, based upon the teachings herein, changes and modifications may be made without departing from this subject matter described herein and its broader aspects and, therefore, the appended claims are to encompass within their scope all such changes and modifications as are within the true spirit and scope of this subject matter described herein. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

1-112. (canceled)

113. A method comprising:
receiving a control command from a patient internal device within a patient at an ingestible device within the patient; and
controlling an operation of the ingestible device based on instructions within the control command.

114. (canceled)

115. The method of claim 113 wherein receiving a control command from a patient internal device within a patient at an ingestible device within the patient comprises:
receiving the control command based on status information associated with the ingestible device and previously provided to the patient internal device.

116. The method of claim 113 wherein receiving a control command from a patient internal device within a patient at an ingestible device within the patient comprises:
receiving the control command based on sensor information associated with the ingestible device and previously provided to the patient internal device.

117-127. (canceled)

128. The method of claim 113 wherein receiving a control command from a patient internal device within a patient at an ingestible device within the patient comprises:
receiving the control command as including a command to the ingestible device to control the operation including releasing medication from the ingestible device.

129. The method of claim 113 wherein receiving a control command from a patient internal device within a patient at an ingestible device within the patient comprises:
receiving the control command as including a command to the ingestible device to control the operation including releasing a specified amount of medication from the ingestible device.

130. The method of claim 113 wherein receiving a control command from a patient internal device within a patient at an ingestible device within the patient comprises:
receiving the control command as including a command to the ingestible device to control the operation including stopping a motion thereof.

131. The method of claim 113 wherein receiving a control command from a patient internal device within a patient at an ingestible device within the patient comprises:
receiving the control command as including a command to the ingestible device to control the operation including designating a location within the patient.

132-145. (canceled)

146. The method of claim 113 wherein receiving a control command from a patient internal device within a patient at an ingestible device within the patient comprises:
receiving the control command from the patient internal device at the ingestible device while the patient internal device and the ingestible device are in wireless communication with one another within the patient, and are spatially separated and mechanically decoupled from one another.

147-157. (canceled)

158. The method of claim 113 wherein controlling an operation of the ingestible device based on instructions within the control command comprises:
controlling the operation including releasing a specified amount of medication from the ingestible device, based on the instructions.

159. The method of claim 113 wherein controlling an operation of the ingestible device based on instructions within the control command comprises:
controlling the operation including stopping a motion of the ingestible device, based on the instructions.

160. The method of claim 113 wherein controlling an operation of the ingestible device based on instructions within the control command comprises:
controlling the operation including designating a location within the patient, based on the instructions.

161-162. (canceled)

163. The method of claim 113 wherein controlling an operation of the ingestible device based on instructions within the control command comprises:
controlling the operation including executing a treatment algorithm to execute a treatment of the patient based thereon, based on the instructions.

164-165. (canceled)

166. A computer program product comprising:
a signal-bearing medium bearing:
(a) one or more instructions for receiving a control command from a patient internal device within a patient at an ingestible device within the patient; and
(b) one or more instructions for controlling an operation of the ingestible device based on instructions within the control command.

167. The computer program product of claim 166, wherein the signal-bearing medium includes a computer-readable medium.

168. The computer program product of claim 166, wherein the signal-bearing medium includes a recordable medium.

169. The computer program product of claim 166, wherein the signal-bearing medium includes a communications medium.

170. A system comprising:
means for receiving a control command from a patient internal device within a patient at an ingestible device within the patient; and
means for controlling an operation of the ingestible device based on instructions within the control command.

171. (canceled)

172. The system of claim 170 wherein means for receiving a control command from a patient internal device within a patient at an ingestible device within the patient comprises:
means for receiving the control command based on status information associated with the ingestible device and previously provided to the patient internal device.

173. The system of claim 170 wherein means for receiving a control command from a patient internal device within a patient at an ingestible device within the patient comprises:
means for receiving the control command based on sensor information associated with the ingestible device and previously provided to the patient internal device.

174. The system of claim 170 wherein means for receiving a control command from a patient internal device within a patient at an ingestible device within the patient comprises:
means for receiving the control command based on a characteristic of a medication associated with the ingestible device, the characteristic having been previously provided to the patient internal device.

175. The system of claim 170 wherein means for receiving a control command from a patient internal device within a patient at an ingestible device within the patient comprises:
means for receiving the control command based on positional information of the ingestible device within the patient, the positional information having been previously provided to the patient internal device.

176. The system of claim 170 wherein means for receiving a control command from a patient internal device within a patient at an ingestible device within the patient comprises:
means for receiving the control command based on patient-external information available at the patient internal device.

177. The system of claim 170 wherein means for receiving a control command from a patient internal device within a patient at an ingestible device within the patient comprises:
means for receiving the control command based on sensor information associated with the patient internal device.

178. The system of claim 170 wherein means for receiving a control command from a patient internal device within a patient at an ingestible device within the patient comprises:
means for receiving the control command based on a sensed condition associated with the patient and sensed using a sensor associated with the patient internal device.

179. The system of claim 170 wherein means for receiving a control command from a patient internal device within a patient at an ingestible device within the patient comprises:
means for receiving the control command based on a condition associated with the patient and sensed using a sensor in communication with the patient internal device.

180. The system of claim 170 wherein means for receiving a control command from a patient internal device within a patient at an ingestible device within the patient comprises:
means for receiving the control command based on a physiological condition of the patient that is sensed using a sensor associated with the patient internal device.

181. (canceled)

182. The system of claim 170 wherein means for receiving a control command from a patient internal device within a patient at an ingestible device within the patient comprises:
means for receiving the control command based on passage of a pre-determined time interval.

183. The system of claim 170 wherein means for receiving a control command from a patient internal device within a patient at an ingestible device within the patient comprises:
means for receiving the control command as including a command to the ingestible device to control the operation including opening a chamber of the ingestible device.

184. The system of claim 170 wherein means for receiving a control command from a patient internal device within a patient at an ingestible device within the patient comprises:
means for receiving the control command as including a command to the ingestible device to control the operation including promoting a degradation of a surface of the ingestible device.

185. The system of claim 170 wherein means for receiving a control command from a patient internal device within a patient at an ingestible device within the patient comprises:
means for receiving the control command as including a command to the ingestible device to control the operation including releasing medication from the ingestible device.

186-191. (canceled)

192. The system of claim 170 wherein means for receiving a control command from a patient internal device within a patient at an ingestible device within the patient comprises:
means for receiving the control command as including a command to the ingestible device to control the operation including executing a treatment algorithm and to execute a treatment of the patient based thereon.

193-194. (canceled)

195. The system of claim 170 wherein means for receiving a control command from a patient internal device within a patient at an ingestible device within the patient comprises:
means for receiving the control command from the patient internal device, the patient internal device being implanted within the patient.

196. The system of claim 170 wherein means for receiving a control command from a patient internal device within a patient at an ingestible device within the patient comprises:
means for receiving the control command from the patient internal device, the patient internal device being inserted within the patient.

197. The system of claim 170 wherein means for receiving a control command from a patient internal device within a patient at an ingestible device within the patient comprises:
means for receiving the control command from the patient internal device, the patient internal device being movable within the patient.

198. The system of claim 170 wherein means for receiving a control command from a patient internal device within a patient at an ingestible device within the patient comprises:
means for receiving the control command from the patient internal device, the patient internal device including a secondary ingestible device within the patient.

199. The system of claim 170 wherein means for receiving a control command from a patient internal device within a patient at an ingestible device within the patient comprises:
means for receiving the control command from the patient internal device, the patient internal device including a secondary ingestible device within the patient having secondary digestion characteristics than the ingestible device.

200. (canceled)

201. The system of claim 170 wherein means for receiving a control command from a patient internal device within a patient at an ingestible device within the patient comprises:
means for receiving the control command from the patient internal device at the ingestible device while the patient internal device and the ingestible device are spatially separated from one another within the patient.

202. The system of claim 170 wherein means for receiving a control command from a patient internal device within a patient at an ingestible device within the patient comprises:
means for receiving the control command from the patient internal device at the ingestible device while the patient internal device and the ingestible device are mechanically decoupled from one another within the patient.

203. The system of claim 170 wherein means for receiving a control command from a patient internal device within a patient at an ingestible device within the patient comprises:
means for receiving the control command from the patient internal device at the ingestible device while the patient internal device and the ingestible device are in wireless communication with one another within the patient, and are spatially separated and mechanically decoupled from one another.

204. The system of claim 170 wherein means for receiving a control command from a patient internal device within a patient at an ingestible device within the patient comprises:
means for receiving the control command by wireless transmission at the ingestible device.

205. (canceled)

206. The system of claim 170 wherein means for controlling an operation of the ingestible device based on instructions within the control command comprises:
means for controlling the operation including means for releasing of at least one medication from the ingestible device, based on the instructions.

207. The system of claim 170 wherein means for controlling an operation of the ingestible device based on instructions within the control command comprises:
means for controlling the operation based on the instructions and in association with at least one secondary control command received from a patient-external device located externally to the patient.

208-212. (canceled)

213. The system of claim 170 wherein means for controlling an operation of the ingestible device based on instructions within the control command comprises:
means for controlling the operation including means for collecting one or more images by the ingestible device, based on the instructions.

214. (canceled)

215. The system of claim 170 wherein means for controlling an operation of the ingestible device based on instructions within the control command comprises:
means for controlling the operation including means for releasing a specified amount of medication from the ingestible device, based on the instructions.

216-222. (canceled)

223. A system comprising:
a computing device including computer-executable instructions that when executed on the computing device, cause the computing device to
receive a control command from a patient internal device within a patient at an ingestible device within the patient; and
control an operation of the ingestible device based on instructions within the control command.

224. The system of claim 223 wherein the computing device comprises:
a memory and a processor implemented in the ingestible device.

225. A system, the system comprising
(a) a receiver configured to receive a control command from a patient internal device within a patient at an ingestible device within the patient; and
(b) control logic configured to control an operation of the ingestible device based on instructions within the control command.

226. The system of claim 225 wherein the receiver is configured to receive the control command at the ingestible device using a wireless communications link.

227. The system of claim 225 wherein the control logic is configured to control the operation of the ingestible device based on sensed data received from a sensor and incorporated into the control command by the patient internal device.

228-311. (canceled)
